(19) United States
(12) Patent Application Publication
(10) Pub. No.: US 2004/0180069 A1
(43) Pub. Date: Sep. 16, 2004

(54) Cosmetic powder on a silica base and method for preparation

(76) Inventors: Loic Bleuez, Saint Laurent du Var (FR); Maryse Porcu, Beausoleil (FR); Karin Golz-Berner, Monaco (MC)

Correspondence Address: Stephan A. Pendorf, Pendorf & Cutliff, 5111 Memorial Highway, Tampa, FL, 33634-7356, US

(21) Appl. No.: 10/795,670

(22) Filed: Mar. 8, 2004

(30) Foreign Application Priority Data
Mar. 13, 2003 (DE) 103 12 124.2

(51) Int. Cl. A61K 38/16; A61K 7/00; A61K 7/06

(52) U.S. Cl. 424/401; 424/74; 514/6

(57) ABSTRACT

The present invention relates to a powder on a silica base showing a markedly improved UV stability and the method of preparation of the powder. The powder of the invention has a content of 1-9% by weight hydrophobic, fine-structured silica with a BET surface within the range of 180-280 m²/g and a mean particle size of 5-30 nm, 20-95% by weight of water, and 0.1-20% by weight of a hydrophilic moisturising substance, with the SiO₂:water ratio being within the range of 1:11 to 1:30, and wherein the powder does not contain any oil, watersoluble PVP, VA acrylic or urethane polymers or hydro colloids. The method of preparation is a mechanic mixing process of a mixture of water and hydrophilic moisturising substance with the silica at 1000-3000 rpm for 3-15 minutes.

### FIELD OF THE INVENTION

The present invention relates to a powder on a silica base showing a markedly improved UV stability and the method of preparation of the powder.

### STATE OF THE ART

A great number of powders are already known. In the recent past, powders with a higher water percentage have increasingly been described. U.S. Pat. No. 6,290,941 discloses powder/liquid compositions containing hydrophobically coated silica with water and a water-soluble polymer being, at the same time, oil-free.

### OBJECT OF THE INVENTION

It is the object of the invention to develop a cosmetic powder which has a stable formulation at very high water percentages and which powder is able to trickle. A further object is a powder which is characterised at the same time by an improved UV stability without the addition of UV filters.

### SUMMARY OF THE INVENTION

According to the invention, the cosmetic powder on a silica base comprises 1-9% by weight of hydrophobic, fine-structured silica (silicon dioxide) with a BET (J. Amer. Chem. Soc. 60(1938)309 ff.) surface within the range of 180-280 m²/g and a mean particle size of 5-30 nm, 20-95% by weight water and 0.1-20% by weight of a hydrophilic moisturising substance with the SiO₂ to water ratio being within the range of 1:11 to 1:30 and where the percentage figures are related to the total weight of the powder which is able to trickle, and wherein the powder does not contain any oil or watersoluble PVP or VA acrylic or urethane polymers or hydro colloids.

The powder according to the invention may have a very high percentage of water but may still have a stable powder formulation in which the powder remains able to trickle. In addition to that, such powder shows a very high UV stability, and even after a prolonged direct sun irradiation water does not separate on the bottom of the box. This allows the manufacture of a more stable end product which makes possible also a mixing with other powdery ingredients, such as cyclodextrin capsules or KSP 100 (Dimethicone/Vinyl Dimethicone Cross Polymer). The powder does not contain any oil and also no water-soluble PVP, VA, acrylic or urethane polymers as in U.S. Pat. No. 6,290,941 with the exception of perfume compounds or aromatic raw materials. The powder does also not contain hydrophilic colloids which are form net-like structures.

The SiO₂:water ratio is preferably within the range of 1:15 to 1:25, in particular 1:11 up to 1:19.

The moisturising substance may be selected from the group consisting of ethylene glycol, butylene glycol, 2,3-butandiol, propylene glycol, dipropylene glycol, tripropylene glycol, hexylene glycol, glycerine, diglycerine, glucose, fructose, lactose, saccharose, maltose, mannitol, mannose, PEG-4 to PEG-800, sorbitol, polyglyceryl sorbitol, urea, xylitol and mixtures thereof. Particular preference is given to PEG-16, glycerine, sorbitol and mixtures thereof.

Preferred percentages of moisturising substances range from 0.1 to 10% by weight.

Preferred water percentages are within the range of 40-95% by weight, preferably 60-95% by weight, in particular 72-95% by weight, specifically 82 to 95% by weight.

The content of hydrophobic, fine-structured silica is preferably within the range of 1 to 7% by weight. The silica is preferably a silica made hydrophobic with a silan-amine with a carbon percentage of 3-4% by weight and a particle size in the range of 5 to 12 nm.

The cosmetic powder according to the present invention may contain other cosmetic ingredients, such as plant extracts, yeast extracts, vitamins, panthenol, coated and uncoated pigments such as glittering and pearly lustre pigments, other powdery ingredients such as bentonite or boron nitride, PMMA such as methyl methacrylate crosspolymer, encapsulated or un-encapsulated perfume oils such as e.g. perfume oils encapsulated in cyclodextrines, preservatives, protective agents, Dimethicone/vinyl Dimethicone Cross Polymer, organic and inorganic UV filters such as cinnamic acid and benzophenone derivatives as well as TiO₂ and ZnO, ethyldiglycol and mixtures thereof.

As yeast extracts, for instance, at least 0.5% by weight of a mixture of enzymes and vitamins containing at least 150 units/ml (U/ml) of superoxide dismutase may be used prepared according to DE 4241154 C1. Such mixture of enzymes and vitamins is preferably a disintegration product of a yeast obtained by ultrasound treatment, the disintegration product containing SOD, protease, vitamin B₂, vitamin B₆, vitamin B₁₂, vitamin D₂ and vitamin E. Preferably it contains at least 150 U/ml SOD, protease and the vitamins B and D with the SOD:protease ratio as international units being at least within the range of 3:1 to 8:1.

As plant extracts, 0.01 to 1% by weight can be used of a complex preparation of active substances with a high radical protection factor (RPF) comprising a product obtained by extraction of the bark of Quebracho blanco and subsequent enzymatic hydrolysis, containing at least 90 percent by weight of proanthocyanidine oligomers and up to 10 percent by weight of gallic acid, linked to microcapsules, further an extract of the silkworm obtained by extraction, containing the peptide Cecropine, amino acids and a vitamin mix, further phospholipids and water. Also contained in the complex preparation can be an ultrasound decomposition product of a yeast containing at least 150 units of superoxide dismutase per ml, and one or more cyclodextrins. The complex preparation can be a product according to WO99/66881 example 1 or 2, or WO 01/26617 example 1.

Further plant extracts are a completely clear watery extract of red apples, green apples or a mixture thereof, where the extract is an extract of the total fruit, or a cosmetic base complex which comprises an encapsulated extract from an aqueous extraction of pineapple fruit and the residue obtained by an aqueous extraction of yogurt.

Other possible ingredients are modified copper-binding proteins wherein the copper in the prosthetic group is withdrawn by a previous exchange reaction or which proteins are prepared without copper by recombinant proceedings which are whitening products. Examples are azurin, ascorbat oxidase, laccase, auracyanin etc. Another ingredients can be milled and transparent glass particles with a mean particle size of 10 to 30 nm and a share of 0.1 to 80% by weight, wherein the glass particles are formed from a molten and after that solidified glass melt. Preferred are glass particles from infrared glasses with a particle size of 10-30 nm.

Vitamins and other scavengers may also be added, such as vitamin C and derivatives thereof, for instance ascorbyl ace-tate, ascorbyl phosphate and ascorbyl palmitate; folic acid and derivatives thereof; water soluble vitamin E and derivatives thereof, such as tocopheryl acetate; flavones or flavonoids; amino acids, such as histidine, glycine, tyrosine, tryptophan and derivatives thereof; carotenoids and carotenes, such as α-carotene, β-carotene; uric acid and derivatives thereof; α-hydroxy acids, such as citric acid, lactic acid, malic acid. Sofar as they are oil-soluble an encapsulation to transform them into powder form is used.

Perfume compounds or aromatic raw materials are mostly oil-soluble products. They also can be widely used after absorption on a powder to be mixed to the emulsion powder. All the classic technical ways to reduce fragrance in powder form can be used and/or mixed such as cyclodextrines for encapsulation, silica, bentonite or zeolite for encapsulation and/or absorption, PMMA or stearic acid as micronized powder or wax compound or allyl methacrylate crosspolymer for absorption and adsorption. Aromatic raw materials are water-soluble or water-miscible. They will be used directly in the water phase. Examples are phenoxy ethyl alcohol, phenyl ethyl alcohol, benzylic alcohol, methyl pyrazine, anisyl alcohol, linalol oxide, maltol, calone, cresol, dimethyl benzyl carbinol, vanillin, ethyl acetyl acetate, ethyl maltol, hexenol-3-cis or ethyl acetate. Also possible to use is some floral water as perfume.

A subject matter of the invention is also a method for the preparation of the cosmetic powder. For this, a first mixture containing at least water and a hydrophilic moisturising substance, is mixed with a hydrophobic, fine-structured silica (silicon dioxide) with a BET surface within the range of 180-280 m²/g and a mean particle size of 5-30 nm in the ratio of SiO₂:H₂O within the range of 1:11 to 1:30 by means of a mechanic mixer and 1000 up to 1500 rpm for a period of 3 to 15 minutes producing a second mixture. Further cosmetic additives may be added either in water before emulsion with silica (as liquids) or to the second powder mixture of silica and water (as powders).

The mixing is preferably carried out over a period of 4 to 9 minutes, for instance with a four-blade propeller mixer at about 1,500 rpm.

The powder according to the present invention causes a very pleasant feeling of freshness on the skin. It is UV-proof and is a booster for UV filters, if any, that are contained therein.

### EXAMPLES

The invention is explained below in greater details by means of examples. All data are given in % by weight unless stated otherwise.

### Example 1

### Mica Water Powder

| Phase A | |
|---|---|
| Water | 17.9 |
| Sorbitol | 1 |
| Preservative | 0.1 |
| Phase B | |
| Sylitate silica* [INCI name] | 1 |
| Phase C | |
| Mica | 80 |
| Ascorbic acid powder | 0.1 |

*Silicon dioxide BET surface 190-250 m²/g, C content 3-4%, mean particle size 7 nm.

Phase A was obtained by mixing the preservative and the moisturising substance one after the other with the water by stirring with a RAYNERI TURBOTEST 33/750P with an 80 mm diameter propeller at about 3,000 rpm for 1 minute. Thereafter, the silica was added and stirred for 10 minutes at about 1,000 rpm. At the end, phase C was added by stirring (about 1 min.)

### Example 2

### Perfume Water Powder

| Phase A | |
|---|---|
| Water | 89.5 |
| Sorbitol | 5 |
| Preservative | 0.5 |
| Phase B | |
| Sylitate silica | 5 |

Preparation was carried out according to example 1.

### Example 3

### Vitamin Water Powder

| Phase A | |
|---|---|
| Water | 72.6 |
| Sorbitol | 20 |
| Preservative | 0.4 |
| Panthenol D-50 | 2 |
| Phase B | |
| Sylitate silica | 5 |

Preparation was carried out according to example 1, without phase C.

### Example 4

### Plant Water Powder

| Phase A | |
|---|---|
| Water | 94.3 |
| Sorbitol | 0.1 |
| Preservative | 0.5 |
| Yeast Extract (DE4241154C1) | 0.1-0.5 |
| Phase B | |
| Sylitate silica | 5 |

Preparation was carried out according to example 1, without phase C.

### Example 5

### Light Tanning Water Powder

| Phase A | |
|---|---|
| Water | 86 |
| Sorbitol | 5 |
| Preservative | 0.5 |
| Coloring agent red | 0.08 |
| Coloring agent yellow | 0.048 |
| Coloring agent green | 3.372 |
| (0.1% in water) | |
| Phase B | |
| Sylitate silica | 5 |

Preparation was carried out according to example 1, without phase C.

### Example 6

### Example for Comparison

The advantageous properties of the powder according to the present invention in respect of UV stability were corroborated by comparative tests.

A composition according to example 2 (sample A) was subjected to radiation in a SUNTEST simulator over a period of 12 hours with short UV light between 296 nm and 400 nm and a 12 μm glass filter and 20° C. in the compartment. The same treatment was given to a composition containing 0% Sorbitol (sample B) and to a composition containing 7% Sorbitol (sample C). The results are listed in table 1.

TABLE 1

UV irradiation

| | after 4 hours | after 8 hours | after 12 hours |
|---|---|---|---|
| Sample A | X | x | □ |
| Sample B | □ | ∘ | ∘ |
| Sample C | X | x | x |

x = completely stable emulsion
□ = low water separation at the bottom of the box
∘ = considerable water separation at the bottom of the box

### Example 7

### Perfume Water Powder II

| Phase A | |
|---|---|
| Water | 83.5 |
| Sorbitol | 5 |
| Preservative | 0.5 |
| Phase B | |
| Sylitate silica | 5 |
| Phase C | |
| Perfume oil | 3 |
| Methyl Methacrylate Cross Polymer | 3 |

Preparation was carried out according to example 1. Phase C was obtained by separately mixing the two ingredients in a powder mixer up to the complete adsorption of the perfume oil to the PMMA powder.

1. A cosmetic powder on a silica base, characterised by a content of 1-9% by weight hydrophobic, fine-structured silica with a BET surface within the range of 180-280 m²/g and a mean particle size of 5-30 nm, 20-95% by weight of water, and 0.1-20% by weight of a hydrophilic moisturising substance, with the SiO₂:water ratio being within the range of 1:11 to 1:30 and all percentage data relating to the total weight of the powder which is trickle, and wherein the powder does not contain any oil or watersoluble PVP or VA acrylic polymers or urethane polymers or hydro colloids.

2. A powder according to claim 1 wherein the SiO₂:water ratio is within the range of 1:11 to 1:25.

3. A powder according to claim 1 wherein the content of hydrophobic, fine-structured silicon dioxide is within the range of 1 to 7% by weight.

4. A powder according to claim 1 wherein it contains further cosmetic ingredients, selected from amongst plant extracts, whitening agents, glass particles, modified copper-binding proteins, vitamins, panthenol, coated and uncoated pigments, dyes, floral waters, water soluble aromatic raw materials, water miscible aromatic raw materials and mixtures thereof.

5. A powder according to claim 1 wherein the hydrophilic moisturising substance is selected from the group consisting of PEG-16, glycerine, sorbitol and mixtures thereof.

6. A powder according to claim 1 wherein the water percentage is within the range of 72-95% by weight, preferably 82-95% by weight.

7. A method for the preparation of a cosmetic powder according to claim 1, wherein a mixture containing at least water and a hydrophilic moisturising substance is mixed with a hydrophobic, fine-structured silica with a BET surface within the range of 180-280 m²/g and a mean particle size of 5-30 nm in the ratio SiO₂:H₂O within the range of 1:11 to 1:30 by means of a mechanical mixer and 1000 to 3000 rpm for a period of 3 to 15 minutes and that further liquid ingredients are added in water before emulsion with silica and powder ingredients are added to the powder mixture of silica and water.

8. A method according to claim 7 wherein the mixing is carried out over a period of 4 to 9 minutes.

9. A method according to claim 7, wherein a fragrance is added as powder transformed perfume compound or as aromatic raw material.
